(19) United States
(12) Patent Application Publication
(10) Pub. No.: US 2014/0316181 A1
(43) Pub. Date: Oct. 23, 2014

(54) PROCESS FOR REMOVING OXYGEN FROM C4-HYDROCARBON STREAMS

(71) Applicant: BASF SE, Ludwigshafen (DE)

(72) Inventors: Gauthier Luc Maurice Averlant, Frankfurt (DE); Alireza Rezai, Mannheim (DE); Sonja Giesa, Darmstadt (DE); Martin Dieterle, Ludwigshafen (DE)

(73) Assignee: BASF SE, Ludwigshafen (DE)

(21) Appl. No.: 14/186,142

(22) Filed: Feb. 21, 2014

Related U.S. Application Data
(60) Provisional application No. 61/767,269, filed on Feb. 21, 2013.

(51) Int. Cl. C07C 7/10

(52) U.S. Cl. CPC C07C 7/10
USPC 585/850

(57) ABSTRACT

In a process for removing oxygen from a C₄-hydrocarbon stream comprising free oxygen by catalytic combustion, in which the hydrocarbon stream comprising free oxygen is reacted by catalytic combustion over a catalyst bed in the presence or absence of free hydrogen to give an oxygen-depleted hydrocarbon stream, the catalytic combustion is carried out continuously, the entry temperature in the catalyst bed is at least 300° C. and the maximum temperature in the catalyst bed is not more than 700° C.

### CROSS-REFERENCE TO RELATED APPLICATIONS

This application claims the benefit of U.S. Provisional Application 61/767,269, filed Feb. 21, 2013, which is incorporated herein by reference.

The invention relates to a process for removing oxygen from C₄-hydrocarbon streams comprising free oxygen.

Hydrocarbon streams which comprise free oxygen and from which the free oxygen should or has to be removed can be obtained in various chemical processes.

For example, free oxygen comprised in a gas stream comprising ethylenically unsaturated hydrocarbons can lead to formation of peroxides which are difficult to handle from a safety point of view.

Butadiene comprising free oxygen can be obtained, for example, by oxidative dehydrogenation of n-butenes (1-butene and/or 2-butene). As starting gas mixture for the oxidative dehydrogenation of n-butenes to butadiene, it is possible to use any mixture comprising n-butenes. For example, it is possible to use a fraction which comprises n-butenes (1-butene and/or 2-butene) as main constituent and has been obtained from the C₄ fraction from a naphtha cracker by removal of butadiene and isobutene. Furthermore, gas mixtures which comprise 1-butene, cis-2-butene, trans-2-butene or mixtures thereof and have been obtained by dimerization of ethylene can also be used as starting gas. Gas mixtures which comprise n-butenes and have been obtained by fluid catalytic cracking (FCC) can also be used as starting gas.

Gas mixtures which comprise n-butenes and are used as starting gas in the oxidative dehydrogenation of n-butenes to butadiene can also be produced by nonoxidative dehydrogenation of gas mixtures comprising n-butane. WO2005/063658 discloses a process for preparing butadiene from n-butane, which comprises the steps
A) provision of an n-butane-comprising feed gas stream a;
B) introduction of the n-butane-comprising feed gas stream a into at least one first dehydrogenation zone and nonoxidative catalytic dehydrogenation of n-butane, giving a product gas stream b comprising n-butane, 1-butene, 2-butene, butadiene, hydrogen, low-boiling secondary constituents and possibly water vapor;
C) introduction of the product gas stream b from the nonoxidative catalytic dehydrogenation and an oxygen-comprising gas into at least one second dehydrogenation zone and oxidative dehydrogenation of 1-butene and 2-butene, giving a product gas stream c which comprises n-butane, 2-butene, butadiene, hydrogen, low-boiling secondary constituents and water vapor and has a higher content of butadiene than the product gas stream b;
D) removal of hydrogen, the low-boiling secondary constituents and water vapor, giving a C₄ product gas stream d which consists essentially of n-butane, 2-butene and butadiene;
E) separation of the C₄ product gas stream d into a recycle stream e1 consisting essentially of n-butane and 2-butene and a stream e2 consisting essentially of butadiene by extractive distillation and recirculation of the stream e1 to the first dehydrogenation zone.

This process utilizes the raw materials particularly effectively. Thus, losses of the raw material n-butane are minimized by recirculation of unreacted n-butane to the dehydrogenation. The coupling of nonoxidative catalytic dehydrogenation and oxidative dehydrogenation results in a high butadiene yield. Compared to the production of butadiene by cracking, the process displays a high selectivity. No coproducts are obtained. The complicated separation of butadiene from the product gas mixture of the cracking process is dispensed with.

WO 2006/075025 describes a process for preparing butadiene from n-butane by nonoxidative, catalytic dehydrogenation of n-butane, subsequent oxidative dehydrogenation and work-up of the product mixture. After the oxidative dehydrogenation, the oxygen remaining in the product gas stream can be removed, for example by reacting it catalytically with hydrogen. A corresponding C₄ product gas stream can comprise from 20 to 80% by volume of butadiene, from 20 to 80% by volume of n-butane, from 5 to 50% by volume of 2-butene and from 0 to 20% by volume of 1-butene and also small amounts of oxygen.

The residual oxygen can have an adverse effect because it can act as initiator for polymerization reactions in downstream process steps. This risk is present in particular in the removal of butadiene by distillation and can there lead to deposits of polymers (formation of “popcorn”) in the extractive distillation column. A removal of oxygen is therefore carried out directly after the oxidative dehydrogenation, generally by means of a catalytic combustion stage, in which oxygen is reacted with the hydrogen comprised in the gas stream in the presence of a catalyst. A reduction of the oxygen content to small traces is achieved in this way. α-Aluminum oxide comprising from 0.01 to 0.1% by weight of platinum and from 0.01 to 0.1% by weight of tin is described as a suitable catalyst. As an alternative, catalysts comprising copper in reduced form are also mentioned.

WO 2010/130610 describes a process for preparing propylene oxide by reacting propene with hydrogen peroxide and separating off the propylene oxide to give a gas mixture comprising propene and oxygen. Hydrogen is added to this gas mixture and the oxygen comprised is at least partly reacted with the hydrogen in the presence of a copper-comprising catalyst. Here, the catalyst comprises from 30 to 80% by weight of copper, calculated as CuO.

WO 2006/050969 describes a process for preparing butadiene from n-butane, in which butane is firstly catalytically hydrogenated to butene, followed by an oxidative dehydrogenation (ODH) to form butadiene. It is indicated that the product gas stream can still comprise small amounts of oxygen and if relatively large amounts of oxygen are present, a catalytic combustion stage in which the oxygen is reacted with the hydrogen comprised in the gas stream in the presence of a catalyst is subsequently carried out. A reduction in the oxygen content down to small traces is said to be achieved in this way. In a simulation example, the stream discharged from the ODH comprises 4.5% by volume of oxygen.

Similar processes are described in DE-A-10 2004 059 356 and DE-A-10 2004 061 514. It is stated in each case that oxygen remaining in the product gas from the oxidative dehydrogenation can be removed by reacting it catalytically with hydrogen.

Apart from “popcorn” formation, the oxygen content in hydrocarbon-comprising gas mixtures, in particular gas mixtures comprising butadiene and oxygen, can lead to deactivation of catalysts, to soot deposits, peroxide formation and to a deterioration in the adsorption properties of solvents in the work-up process.

In the preparation of butadiene from n-butane in particular, selective removal of oxygen is a basic prerequisite for the process to be able to be carried out economically, since any loss of the target product butadiene is associated with increased costs.

### A BRIEF DESCRIPTION OF THE FIGURES

FIG. 1 schematically shows the structure of a reactor.

FIG. 2 shows the axial temperature profiles determined for various entry temperatures.

FIG. 3 schematically shows the structure of a reactor.

FIG. 4 shows the axial temperature profiles determined for various wall temperatures.

FIG. 5 shows results for testing detailed below.

FIG. 6 shows results for testing detailed below.

### DETAILED DESCRIPTION OF THE INVENTION

It is an object of the present invention to provide a process for removing oxygen from a C₄-hydrocarbon stream comprising free oxygen by catalytic combustion, in which a residual oxygen content of less than 100 ppm or less than 80 ppm or less than 50 ppm or less than 30 ppm or less than 10 ppm or less than 1 ppm or 0 ppm can be obtained and very little C₄-hydrocarbon as product of value is preferably consumed.

The residual oxygen content should particularly preferably be less than 50 ppm. It is determined by means of electrochemical oxygen sensors such as KE 25 from Figgres or A-3, B-1 or B-3 from Teledyne. After calibration, a measurement accuracy of about 10 ppm of O₂ is achieved.

The object is achieved according to the invention by a process for removing oxygen from a C₄-hydrocarbon stream comprising free oxygen by catalytic combustion, in which the hydrocarbon stream comprising free oxygen is reacted by catalytic combustion over a catalyst bed in the presence or absence of free hydrogen to give an oxygen-depleted hydrocarbon stream, wherein the catalytic combustion is carried out continuously, the entry temperature in the catalyst bed is at least 300° C. and the maximum temperature in the catalyst bed is not more than 700° C.

For the purposes of the invention, the term “C₄-hydrocarbon stream” refers to a hydrocarbon stream in which at least 60% by volume, preferably at least 80% by volume, in particular at least 95% by volume, of the hydrocarbons are C₄-hydrocarbons.

The C₄-hydrocarbon stream preferably originates from the dehydrogenation of butane or dehydrogenation of butene, in particular from the oxydehydrogenation of butene to butadiene. It preferably comprises from 0.5 to 8.0% by volume of free oxygen, more preferably from 1.0 to 8.0% by volume, particularly preferably from 2.0 to 7.0% by volume, in particular from 3.0 to 6.5% by volume.

In an embodiment of the invention, the hydrocarbon stream comprising free oxygen comprises an amount of free hydrogen which is sufficient for reaction with the free oxygen and/or has this added to it, and the free oxygen is reacted with the free hydrogen.

As an alternative, the hydrocarbon stream comprising free oxygen does not comprise any free hydrogen and no free hydrogen is added to it.

In this case, the free oxygen can preferably be reacted with hydrocarbon comprised in the hydrocarbon stream comprising free oxygen or with added methanol, natural gas and/or synthesis gas as reducing agent.

In an embodiment of the invention, the C₄-hydrocarbon stream used according to the invention is obtained according to the following steps:
provision of an n-butane-comprising feed gas stream a;
introduction of the n-butane-comprising feed gas stream a into at least one first dehydrogenation zone and nonoxidative, catalytic dehydrogenation of n-butane, giving a gas stream b comprising n-butane, 1-butene, 2-butenes, butadiene, hydrogen, possibly water vapor, possibly carbon oxides and possibly inert gases;
introduction of a stream f which comprises butane, butenes, butadiene and has been obtained from the gas stream b, and of an oxygen-comprising gas, into at least one second dehydrogenation zone and oxidative dehydrogenation of 1-butene and 2-butenes, giving a gas stream g comprising n-butane, unreacted 1-butene and 2-butenes, butadiene, water vapor, possibly carbon oxides, possibly hydrogen and possibly inert gases, and
removal of the residual oxygen comprised in the gas stream g by means of catalytic combustion to give an oxygen-depleted stream h.

For a description of the dehydrogenation of butane and oxydehydrogenation, reference may be made to the documents indicated at the outset, in particular DE-A-10 2004 059 356 (WO 2006/061202), DE-A-10 2004 061 514 (WO 2006/066848), WO 2010/130610, WO 2006/050969, DE-A-10 2005 002 127 (WO 2006/075025).

The product gas stream leaving the oxidative dehydrogenation comprises not only butadiene and n-butane which has not been separated off but also hydrogen, carbon oxides, oxygen and water vapor. It can further comprise inert gas such as nitrogen, methane, ethane, ethene, propane and propene and also oxygen-comprising hydrocarbons, known as oxygenates, as secondary constituents.

In general, the product gas stream leaving the oxidative dehydrogenation comprises from 2 to 40% by volume of butadiene, from 5 to 80% by volume of n-butane, from 0 to 15% by volume of 2-butenes, from 0 to 5% by volume of 1-butene, from 5 to 70% by volume of water vapor, from 0 to 10% by volume of low-boiling hydrocarbons (methane, ethane, ethene, propane and propene), from 0.1 to 15% by volume of hydrogen, from 0 to 70% by volume of inert gas, from 0 to 10% by volume of carbon oxides, from 0 to 10% by volume of oxygen and from 0 to 10% by volume of oxygenates, where the total amount of the constituents is 100% by volume. Oxygenates can be, for example, furan, acetic acid, methacrolein, maleic anhydride, maleic acid, phthalic anhydride, propionic acid, acetaldehyde, acrolein, formaldehyde, formic acid, benzaldehyde, benzoic acid and butyraldehyde. Acetylene, propyne and 1,2-butadiene can also be comprised in traces.

Other sources of the C₄-hydrocarbon comprising free oxygen are, for example, raffinate II and products of ethylene dimerization.

If the product gas stream comprises more than only minor traces of oxygen, the process stage according to the invention for removing residual oxygen from the product gas stream is carried out. The residual oxygen can have an adverse effect because it can, for example in the case of butadiene, bring about butadiene peroxide formation and act as initiator for polymerization reactions in downstream process steps.

In the case of butadiene production, the removal of oxygen is preferably carried out directly after the oxidative dehydrogenation.

The C₄-hydrocarbon stream comprising free oxygen can comprise an amount of free hydrogen which is sufficient for reaction with the free oxygen. Deficit amounts or the total amount of the free hydrogen required can be added to the hydrocarbon stream. In this way of carrying out the reaction, the free oxygen can be reacted with the free hydrogen, so that only a very small proportion of the hydrocarbon is reacted with the oxygen. Despite the presence of hydrogen, barely any hydrogenation of the hydrocarbon occurs according to the invention.

In an alternative embodiment, the hydrocarbon stream comprising free oxygen does not contain any free hydrogen and no free hydrogen is added to it either. In this case, the free oxygen can be reacted with the hydrocarbon comprised in the hydrocarbon stream comprising free oxygen or with added methanol, natural gas and/or synthesis gas as reducing agent.

The process regime here may be isothermal or adiabatic. An advantage of reacting the hydrogen is the formulation of water as reaction product. The water formed can be easily removed by condensation.

In addition, a low reaction pressure can be advantageous, since a separate compression step, e.g. after the oxidative dehydrogenation, can be avoided in this way. A lower reaction pressure allows a less costly manufacture of the reactor and is advantageous for safety reasons.

The process of the invention is therefore preferably carried out at an absolute pressure of from 0.5 to 20 bar, preferably from 0.9 to 10 bar, particularly preferably from 0.9 to 5 bar, more preferably from 0.9 to 3 bar, in particular from 0.9 to 2 bar.

The reaction is preferably carried out at an entry temperature in the catalyst bed of from 300 to 450° C., particularly preferably from 320 to 400° C. This temperature applies particularly at an oxygen content of from 1 to 3.5% by volume. At higher oxygen contents (e.g. 8% by volume), intermediate cooling can be necessary to adhere to the temperatures according to the claims.

The maximum temperature in the catalyst bed is preferably not more than 650° C. It is preferably in the range from 500 to 650° C., in particular from 580 to 650° C.

If the reaction temperature is too low, it is possible for, for example, butadiene to be hydrogenated. If it is too high, cracking processes can occur.

The reactor type is not restricted according to the invention. For example, the reaction can be carried out in a fluidized bed, in a tray furnace, in a fixed tube reactor or shell-and-tube reactor or in a plate heat exchanger reactor. The fixed-bed catalyst can be operated adiabatically in the industry. The flow through the bed can be either axial or radial. A radial reactor could be advantageous for large volume flows. Owing to the high outlet temperature to be expected (up to 600° C.), feeding from the outside inward can be advantageous. A flow from the inside outward nevertheless gives smaller pressure drops. Monolith reactors can be used for reactions which require little catalyst, advantageously as an adiabatic reactor. Since the residual oxygen concentration should be low, a backmixed system should be avoided. The reactor concept of a tube reactor (fixed-bed reactor) has therefore been found to be useful. Cascading of backmixed fluidized-bed reactors or the use of tray reactors would likewise be conceivable.

Structuring of a fixed bed of the catalyst by means of inert material enables the temperature profile to be adapted and the maximum temperature to be kept in the optimal range.

If a substoichiometric amount of hydrogen is used in the process of the invention, the reaction with hydrogen can serve to reach a sufficiently high temperature for the required reaction between hydrocarbons and oxygen.

If no hydrogen, or a substoichiometric amount of hydrogen, is used, the oxygen reacts predominantly with the most reactive molecule, for example butadiene. Formation of carbon oxides and water occurs as a result. Since the reaction of oxygen with the hydrocarbons proceeds more slowly at low temperature than with hydrogen, the hydrogen is firstly completely consumed.

A further embodiment of the invention comprises carrying out this catalytic reaction together with an oxidative dehydrogenation in a reactor comprising 2 catalysts and optionally intermediate introduction of the combustion gas downstream of the dehydrogenation bed.

According to the invention, the term “catalyst bed” refers to the region of a reactor in which the catalyst is present as a fixed-bed catalyst. It can be one catalyst bed, one or more catalyst monoliths or other structured packings.

The reactor used for the catalytic combustion, through which continuous flow occurs, optionally firstly comprises a bed of inert material which allows heating of the gases to be used. This is followed by the catalyst bed. The entry temperature in the catalyst bed relates to the region of the catalyst bed into which the gas mixture to be reacted enters.

The catalytic combustion can be carried out over any suitable catalysts, as are also described, for example, in the abovementioned prior art, in particular in WO 2006/061202.

According to the invention, preference is given to using a catalyst which comprises at least one noble metal and/or at least one transition metal on a support.

Possible noble metals are, in particular, Pt, Pd, Ir, Rh, Ru, Au and Ag and mixtures thereof.

Suitable transition metals are preferably those of groups 7 to 14 of the Periodic Table of the Elements, particularly preferably Mn, Fe, Ni, Co, Cu, Zn, Sn. Particular preference is given to using Sn.

As noble metal, preference is given to using platinum or a platinum-comprising alloy.

Particular preference is given to the combination of platinum and tin as active metals. Here, the proportion of platinum, based on the total catalyst, is preferably from 0.01 to 1% by weight, particularly preferably from 0.02 to 0.5% by weight, in particular from 0.05 to 0.2% by weight.

Tin is likewise preferably used in an amount, based on the total catalyst, of from 0.01 to 1% by weight, particularly preferably from 0.02 to 0.5% by weight, in particular from 0.05 to 0.2% by weight.

The weight ratio of tin to platinum is preferably from 1:4 to 4:1, particularly preferably from 1:2 to 2:1, in particular about 1:1.

Apart from platinum and tin, it is possible for alkali metal compounds and/or alkaline earth metal compounds optionally to be concomitantly used in amounts of <2% by weight, in particular <0.5% by weight, based on the total catalyst. Particular preference is given to the catalyst comprising exclusively platinum and tin as active metals.

As catalyst supports, it is possible to use any suitable solid support materials. The support is preferably a-aluminum oxide or zeolite A, in particular a-aluminum oxide. It preferably has a BET surface area of from 0.5 to 15 m²/g, more preferably from 2 to 14 m²/g, in particular from 7 to 11 m²/g. A shaped body is preferably used as support. Preferred geometries are, for example, pellets, annular pellets, spheres, cylinders, star extrudates or cogwheel-shaped extrudates having diameters of from 1 to 10 mm, preferably from 2 to 6 mm. Particular preference is given to spheres or cylinders, in particular spheres.

When this catalyst is used, the butadiene loss can be suppressed and at the same time the residual oxygen can be reliably removed when starting out from a butadiene-comprising C₄-hydrocarbon stream. When the catalyst is used in the temperature range according to the invention, a low level of secondary reactions occurs and the reaction can be carried out using an excess of hydrogen, a substoichiometric amount of hydrogen or in the absence of hydrogen.

The reaction is alternatively carried out over a catalyst comprising from 0.01 to 0.5% by weight of platinum, based on the catalyst, and optionally tin on zeolite A as support, with the weight ratio of Sn:Pt being from 0 to 10.

This catalyst preferably comprises zeolite A as support. Based on the support, preferably at least 80% by weight, particularly preferably at least 90% by weight, in particular at least 95% by weight, of zeolite A is present in the support. In particular, the support is made up entirely of zeolite A.

Zeolite A is a synthetic, crystalline aluminosilicate and in its hydrated sodium form has the empirical formula Na₁₂((AlO₂)₁₂(SiO₂)₁₂)×27 H₂O. The term “zeolite A” comprises various variants of this compound which all have the same aluminosilicate lattice. However, they can comprise other ions such as potassium or calcium instead of sodium ions. Low-water or water-free forms are also counted as zeolite A according to the invention. Other names are molecular sieve A, LTA (Linde type A), MS 5 A (with Ca), MS 4 A (with Na), NF3 A (with K), Sasil®.

Zeolite A has a framework structure made up of AlO₄ and SiO₄ tetrahedra. They form a covalent lattice with voids which generally comprise water. AlO₄ and SiO₄ tetrahedra are present in a ratio of 1:1. Here, aluminum and silicon atoms are alternately connected via oxygen atoms.

Overall, the lattice has a negative charge which is balanced by ionic compounds having cations such as sodium ions. As three-dimensional structure, zeolite A has a sodalite cage.

This catalyst preferably comprises from 0.01 to 0.5% by weight, preferably from 0.05 to 0.4% by weight, in particular from 0.1 to 0.3% by weight of platinum, based on the catalyst. It can additionally comprise tin, with the weight ratio of Sn:Pt being from 0 to 10, preferably from 0 to 7, particularly preferably from 0 to 3. When tin is concomitantly used, the weight ratio of Sn:Pt is preferably from 0.5 to 10, particularly preferably from 0.7 to 4, in particular from 0.9 to 1.1. Especial preference is given to a weight ratio of Sn:Pt of 1:1.

This catalyst can preferably also comprise further metals, for example alkali metal compounds and/or alkaline earth metal compounds, preferably in amounts of <2% by weight, in particular <0.5% by weight, based on the catalyst, in addition to platinum and tin. Particular preference is given to the catalyst comprising exclusively platinum and optionally tin as active metals.

In the finished catalyst, the BET surface area is preferably from 10 to 80 m²/g, particularly preferably from 15 to 50 m²/g, in particular from 20 to 40 m²/g.

The catalyst can be used in any suitable form. It is preferably used as shaped bodies having an average diameter in the range from 1 to 10 mm, particularly preferably from 2 to 8 mm, in particular from 2.5 to 5 mm. The shaped body can have any suitable shape; it can be present as extrudate, pellet, granules, crushed material or preferably in spherical form having the average diameter indicated. Further possible shaped bodies are annular pellets, cylinders, star extrudates or cogwheel-shaped extrudates.

As an alternative, the catalysts mentioned can be present as monolith, with the monolith being able to have the catalyst as washcoat on a support structure. This support structure can prescribe the three-dimensional structure of the monolith. For example, the support structure can be made up of cordierite.

The proportion of washcoat in the total monolith is preferably from 0.5 to 5 g/inch³.

The catalyst can be produced by any suitable processes. It is preferably produced by impregnation of the support with a solution of a platinum compound and optionally a tin compound and subsequent drying and calcination. For example, platinum nitrate can be used as aqueous solution for impregnating the support. Impregnation can be followed by drying, preferably at from 80 to 150° C., and calcination, preferably at from 200 to 500° C. Drying is preferably carried out for a period in the range from 1 to 100 hours, particularly preferably from 5 to 20 hours. Calcination is preferably carried out for a period of from 1 to 20 hours, particularly preferably from 2 to 10 hours.

The actual production of the catalyst can be followed by a silylation, for example using an aqueous colloidal dispersion of very small silicon dioxide particles, as are available, for example, under the name Ludos® from Helm AG. This silylation, too, can be carried out by impregnation with subsequent drying and calcination, as described above.

The catalyst used according to the invention has, in particular, long-term stability, especially in the dehydrogenation of butane or butene to produce butadiene, where free oxygen is to be separated off from the butadiene-comprising product stream.

The catalyst which is preferably used has the advantage that it catalyzes, in particular, the reaction of hydrogen with oxygen without appreciable reaction of hydrocarbon with the free oxygen occurring. In the case of the preparation of butadiene from butene or n-butane, reaction of the butadiene with the free oxygen preferably does not occur.

A further advantage of the use of the catalyst according to the invention is its stability in the presence of water in the feed, in particular at from 5 to 30% of water in the feed.

The preparation of butadiene from n-butane is, for example, carried out by introducing an n-butane-comprising feed gas stream in at least one first dehydrogenation zone and carrying out nonoxidative catalytic dehydrogenation of the n-butane, giving a product gas stream comprising n-butane, 1-butene, 2-butene, butadiene, hydrogen, low-boiling secondary constituents, possibly carbon oxides and possibly water vapor. This product gas stream is fed together with an oxygen-comprising gas into at least one second dehydrogenation zone for oxidative dehydrogenation, giving a product gas stream comprising n-butane, 2-butenes, butadienes, low-boiling secondary constituents, carbon oxides and water vapor.

The invention is illustrated by the following examples.

### EXAMPLES

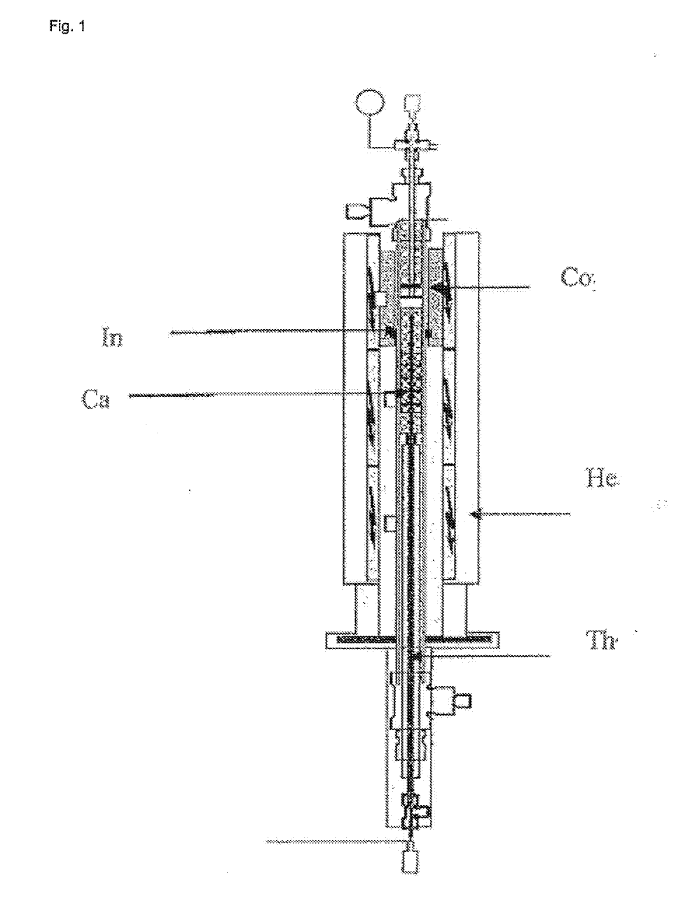

The catalytic removal of oxygen was examined in an adiabatic reactor. FIG. 1 schematically shows the structure of the reactor whose dimensions are listed below:

| | | |
|---|---|---|
| Length: | 200 | cm |
| External diameter: | 2.5 | cm |
| Wall thickness: | 0.2 | cm |
| Internal diameter: | 2.1 | cm |
| External diameter of the thermocouple sheath: | 3.1 | mm |

| | |
|---|---|
| Material: | steel (1.4841) |

The symbols in FIG. 1 have the following meanings:

In: Inert material

Co: Copper block

Ca: Catalyst

He: Heating element in zone 3

Th: Thermocouple

The reactor consists essentially of 2 zones. In the first heating zone, an inert bed is preheated to the desired temperature by means of an accompanying heating element. Between the accompanying heating element and the reactor there is a copper block in order to make homogeneous distribution of the heat in the first zone possible. The catalyst is installed in the second zone. The reactor is in this section surrounded by insulation material in order to keep the heat loss low. Two accompanying heating elements were also installed in the insulation material in order to allow temperature equilibration between the interior of the reactor and the outside.

In the middle of the reactor, there is a thermocouple sheath in which thermocouples are placed. These thermocouples make it possible to record an axial temperature profile in the catalytic bed. A pneumatically operated, multiple thermocouple having four measurement points was used for determining the temperature profiles with a resolution of 2 cm in the catalyst bed. The catalyst bed was packed between an inert material (steatite) which served as guard bed.

The gas flows through the reactor from the top downward.

The reactor was operated under the following typical conditions:

| | |
|---|---|
| Catalyst volume: | 75 ml |
| Mass of catalyst: | 54.6 g |
| Inlet temperature: | 150-450° C. |
| Outlet pressure: | 1.5-2.5 bara |
| GHSV: | 10 000-12 000 standard l of gas l of cat⁻¹ h⁻¹ |
| Entry concentration of | 3% by volume |
| oxygen: | |
| Ratio of hydrogen/oxygen: | 2.1-2.5% |
| Hydrocarbon concentration: | about 20% by volume |
| Water concentration: | about 13% by volume |
| Balance: | nitrogen |

Production of the Catalyst

The catalyst comprises 99.7% by weight of zeolite A, molecular sieve 3A (from Roth GmbH), 0.3 mm type 562 C, bead form, spheres having a diameter in the range from 2.5 to 5 mm, and 0.3% by weight of platinum.

1000 g of molecular sieve and 5.2 g of platinum nitrate are used for producing the catalyst. Platinum nitrate is dissolved in water and the solution is made up to a total solution volume of 460 ml. The support is then impregnated to 100% of its water absorption. For this purpose, the molecular sieve was divided among two porcelain dishes, the impregnation solution was divided and the mixtures were mixed well.

This was followed by drying at 120° C. for 16 hours in a convection drying oven and calcination at 400° C. for four hours in a muffle furnace.

To carry out the silylation, the catalyst obtained in this way was placed in a glass beaker and a solution of Ludox and water in a ratio of 1:10 (final concentration: 4% by weight) was produced. The amount was selected so that the catalyst in the glass beaker could be well covered. The mixture was stirred at regular intervals and filtered through a fluted filter after 40 minutes. This was once again followed by drying at 120° C. for 16 hours in a convection drying oven and subsequent calcination at 400° C. for 4 hours in a muffle furnace.

Elemental analysis indicated a proportion of Pt in the catalyst of 0.27% by weight.

The experiments using this catalyst show that the removal of oxygen is a fast reaction and can thus be operated at high loads (up to 11 000 standard l of gas/l of cat/h⁻¹). The specification of 100 ppm can be met at entry temperatures in the catalyst bed of greater than or equal to 290° C. for a GHSV of about 11 000 h⁻¹. If the temperature is too low (<290° C.), the hydrocarbons present in the feed stream are reacted significantly. For example, at 290° C. the conversion of the total hydrocarbons is about 4%. The main products here are butene (selectivity over 75%) and COₓ. If the temperature at the reactor inlet is increased, the specification for O₂ is still achieved, but the conversion of the hydrocarbons decreases significantly (2% at about 350° C.).

Temperature Conditions:

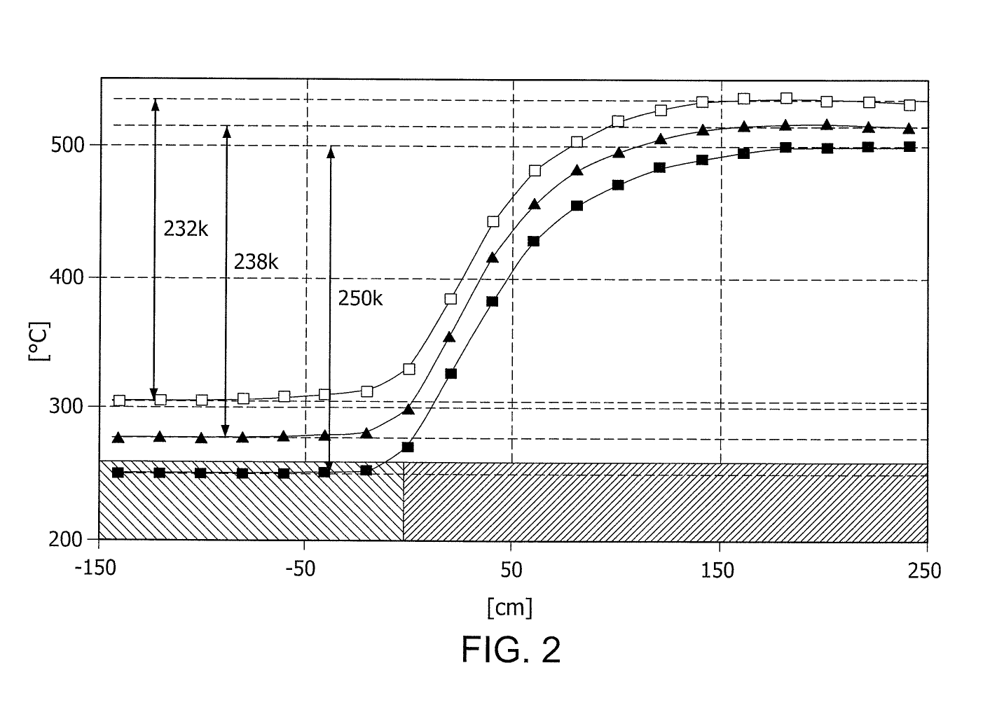

FIG. 2 shows the axial temperature profiles determined for various entry temperatures. The temperature in ° C. is plotted over the length of the (catalyst) bed in cm. Catalyst is present from length zero. The oxygen content in the feed stream was 3% by volume, the molar ratio of hydrogen to oxygen was 2.6, the GHSV was about 11 000 h⁻¹ and the temperature of the heating sleeves was about 496° C. The O₂ specification was met in every experiment and this is reflected in the temperature increase, which is almost identical for all experiments. It also corresponds to the adiabatic temperature increase. It can be seen that the temperature increase has reached 90% of the maximum temperature increase after only half the bed length. If the residual oxygen specification is to be less than 100 ppm, the GHSV could be increased further.

Without Hydrogen:

As an alternative to the process using hydrogen, the oxygen content in the offgas stream can be reduced by catalytic reaction with the hydrocarbons present in the gas. The oxygen will react predominantly with the most reactive molecule, i.e. in this case butadiene, and leads to the formation of CO₂ and H₂O. The reaction of O₂ with the hydrocarbons is slower at low temperature than with hydrogen. This reaction should therefore preferably be carried out at lower space velocities over the catalyst and/or at higher temperature (compared to the mode of operation with hydrogen). However, rapid reaction of the O₂ appears to inhibit soot formation, so that relatively high temperatures would be preferred. At relatively high temperatures, this reaction has a rate comparable to the H₂/O₂ reaction and can similarly be carried out at high loads (experiment with GHSV =10 500 h⁻¹, inlet temperature: 400° C.).

Hybrid Mode of Operation:

Should a gas stream comprise H₂ (e.g. from the BDH stage), it could be introduced into the O₂ removal stage for the purpose of removing O₂. Oxygen will preferentially react with hydrogen at relatively low temperatures. If hydrogen is present in a substoichiometric amount, the remaining O₂ reacts further with the hydrocarbons. The reaction with H₂ can also serve to achieve a sufficiently high temperature for the reaction between the hydrocarbons and oxygen (ignition). For example, the ODH stage is, depending on the catalyst used, operated in the range from 320 to 420° C. Should this stage be operated at a low temperature, a heat exchanger between the ODH and O₂ removal stages would be advantageous in order to bring the gas mixture to the desired temperature. However, experience shows that a mixture of butadiene and oxygen tends to form polymer-like deposits at temperatures above 250° C. For this reason, a rapid increase in temperature with rapid degradation of O₂ is desirable. For this purpose, hydrogen can be introduced in such a way that a sufficiently high temperature for the catalytic combustion of butadiene with O₂ is achieved. Operation of an additional heat exchanger is saved in the process and the risk of blockage of the plant is reduced thereby.

### Example 2

Removal of Oxygen Using Hydrogen:

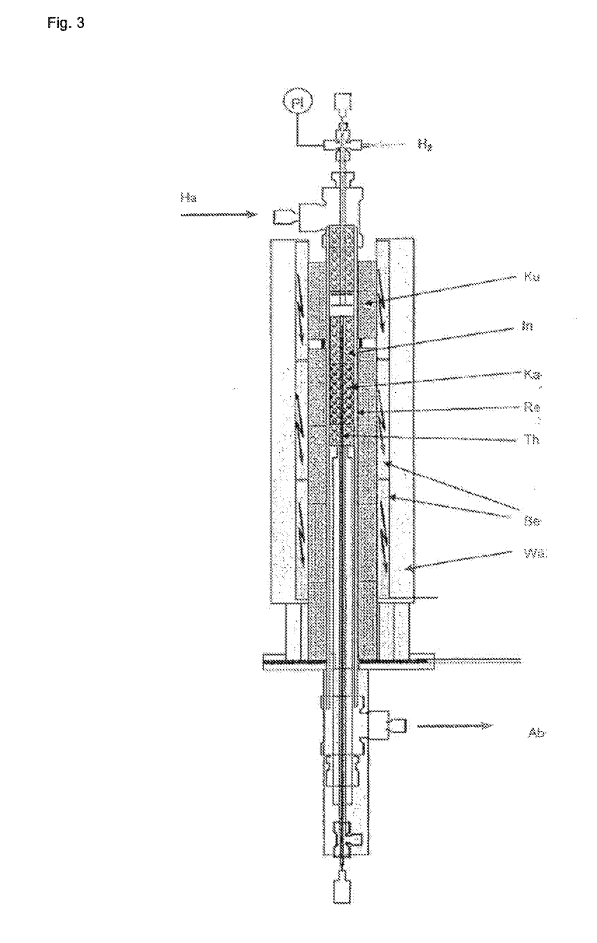

The catalytic removal of oxygen was examined in a wall-cooled reactor. FIG. 3 schematically shows the structure of the reactor, and its dimensions are listed as follows:

| | | |
|---|---|---|
| Length: | 200 | cm |
| External diameter: | 2.5 | cm |
| Wall thickness: | 0.2 | cm |
| Internal diameter: | 2.1 | cm |
| External diameter of the thermocouple sheath: | 3.1 | mm |

| | |
|---|---|
| Material: | steel (1.4841) |

In FIG. 3, the symbols have the following meanings:

HA: Main stream

Ku: Copper blocks

In: Inert bed

Ka: Catalyst

Re: Reactor wall

Th: Thermocouple sheath

Be: Heating

Wa: Thermal insulation

Ab: Offgas stream

The reactor consists of 3 heating zones and is provided with copper blocks to enable a uniform temperature field to be set at the reactor wall. In the first heating zone, an inert bed is preheated to the desired temperature. In the second heating zone, the wall temperature of the catalytic bed is set.

In the middle of the reactor there is a thermocouple sheath in which thermocouples are placed. These thermocouples make it possible to record an axial temperature profile in the catalytic bed. A pneumatically operated, multiple thermocouple having four measurement points was used for determining the temperature profiles with a resolution of 2 cm in the catalyst bed. The catalyst bed was packed between an inert material (steatite) which served as guard bed.

The reactor was operated under the following typical conditions:

| | |
|---|---|
| catalyst volume: | 0.05-0.1 l |
| mass of catalyst: | 0.010-0.1 kg |
| inlet temperature: | 150-450° C. |
| outlet pressure: | 1.5-2.5 bara |
| GHSV: | 2000-12 000 standard l of gas l of cat⁻¹ h⁻¹ |
| oxygen entry concentration: | 3% by volume |
| ratio of hydrogen/oxygen: | 2.1-2.5% |
| hydrocarbon concentration: | about 20% by volume |
| water concentration: | about 13% by volume |
| balance: | nitrogen |

Production of the Catalyst:

The catalyst comprises 99.7% by weight of zeolite A, molecular sieve 3A (from Roth GmbH), 0.3 mm type 562 C, bead shape, spheres having a diameter in the range from 2.5 to 5 mm, and 0.3% by weight of platinum and was produced as described in example 1.

The experiments using this catalyst showed that the removal of oxygen is a fast reaction and can thus be operated at high loads (up to 11 000 standard l of gas/l of cat/h⁻¹). The specification of 100 ppm can be met at entry temperatures in the catalyst bed greater than or equal to 320° C. If the temperature is too low (<300° C.), up to 12% of the hydrocarbons are reacted. Under these conditions, the reaction is predominantly a hydrogenation of butadiene to butene. For this reason, an isothermal mode of operation is less desirable at a low temperature level. If the entry temperature is increased to above 380° C., H₂ reacts to an extent of more than 90% with O₂, so that the butadiene conversion is kept low. This low conversion is also promoted by a low excess of hydrogen being selected and the residence time over the catalyst being kept short.

Temperature Conditions:

Suitable wall-cooled reactors frequently occur in the chemical industry. In the case of fixed-bed reactors, shell-and-tube reactors in which the tubes are filled with the catalyst and the heat evolved by the reaction is removed by means of a cooling medium in the outer space can be used with preference. For temperatures above 300° C., salt bath reactors are particularly suitable. However, the salt is generally subject to gradual decomposition at temperatures above 460° C. This determines a temperature window in which the process is preferably operated.

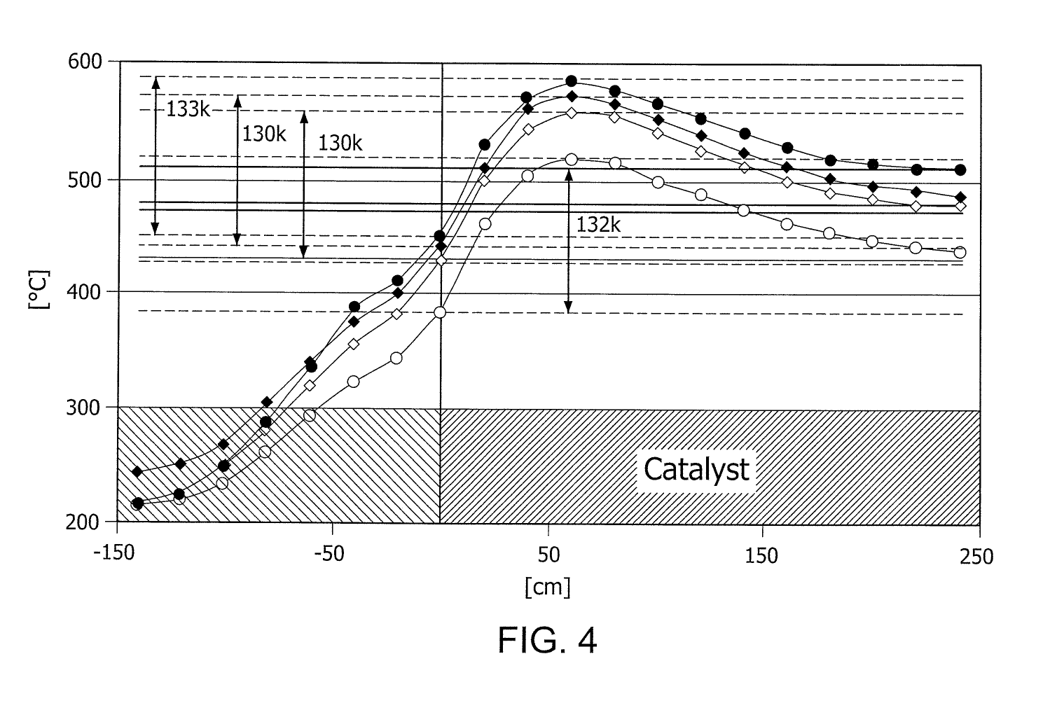

FIG. 4 shows the axial temperature profiles determined for various wall temperatures. The temperature in ° C. is plotted against the length of the (catalyst) bed in cm. The catalyst is present from length 0. The oxygen content in the inlet stream is 3.1% by volume, the molar ratio of hydrogen to oxygen is 2.1 and the GHSV is about 10 500 h⁻¹. Furthermore, the respective wall temperature and also the maximum temperature difference between inlet temperature in the catalyst bed and maximum temperature are plotted. In each experiment, the 02 specification was met and the hydrocarbon conversion was less than 2%.

Independently of the wall temperature, the temperature increase, defined as the difference between the maximum temperature at the hot spot and the entry temperature in the catalyst bed, is virtually identical and in this case corresponds to more than half the adiabatic temperature increase (3% of O₂ correspond to an adiabatic temperature increase of about 250 K). The position of the hot spot remains unchanged in all experiments, which indicates that higher temperatures do not significantly accelerate the reaction between H₂ and O₂. This means that relatively high temperatures (>380° C.) do not significantly influence the course of the reaction and strict control of the height of the hot spot is not absolutely necessary. Nevertheless, the maximum temperature required is preferably set at 600° C. in order for the catalyst not to be subjected to thermal stress.

The height of the hot spot can be influenced by various parameters, e.g. the flow velocity, dilution of the catalyst and the tube diameter. In addition, it is known that heat transport is subject to a resistance between the bed and the wall, so that temperature gradients of more than 30° C. are routine. The temperature at the wall is therefore significantly lower than at the hot spot, which represents an advantage for the salt of a brine bath heat exchanger. The use of a salt bath reactor is therefore possible.

The ODH stage is, depending on the catalyst used, preferably operated at temperatures in the range from 320 to 420° C. in a salt bath reactor. Previous experiments have shown that the hydrogen is only partially reacted over the ODH catalyst. It is thus possible to couple the removal of oxygen with the ODH stage by introducing hydrogen at the reactor inlet. Since the removal of O₂ by means of H₂ is a fast reaction, the increase in length of the tubes is less than 1 m. To achieve the minimum temperature of 380° C., a two-zone salt bath reactor (with two different salt bath temperatures) can be used.

Without Hydrogen:

As an alternative to the process using hydrogen, the oxygen content in the offgas stream can be reduced by catalytic reaction with the hydrocarbons present in the gas. The oxygen will react predominantly with the most reactive molecule, i.e. in this case butadiene, and leads to the formation of CO₂ and H₂O. At low temperature, the reaction of O₂ with the hydrocarbons is slower than with hydrogen. This reaction should therefore be carried out at relatively low space velocities over the catalyst bed and/or at relatively high temperature (compared to operation using H₂). However, rapid reaction of O₂ appears to inhibit soot formation, so that relatively high temperatures would be preferred. At high temperatures, this reaction has a comparable rate to the H₂/O₂ reaction and can similarly be carried out at high loads in a wall-cooled reactor.

(Experiment with GHSV=10 500 h⁻¹, inlet temperature 400° C.). The sequential arrangement of the ODH stage and the O₂ removal in a single salt bath reactor in which the minimum required temperature prevails in the second zone is possible.

Hybrid Mode of Operation:

Should a gas stream comprise H₂ (e.g. from the BDH stage), it could be introduced into the O₂ removal stage for the purpose of removing O₂. Oxygen will react preferentially with hydrogen at relatively low temperatures. If hydrogen is present in a substoichiometric amount, the remaining O₂ reacts further with the hydrocarbons. The reaction with H₂ can also serve to achieve a sufficiently high temperature for the reaction between the hydrocarbons and oxygen (ignition).

### Example 3

### Influence of the Temperature on the Selectivities

In the reactor as described in example 1, a gas stream consisting of 3% of O₂, 14% of butadiene, 5.5% of butane, 12% of water vapor with balance nitrogen is introduced into the reactor. The catalyst bed consists of 75 ml of undiluted catalyst (DA301 with Pd). The total volume flow is 800 standard l/h. The inlet temperature in the catalyst bed was varied in the range from 170 to 350° C. The admission pressure was kept constant at 1.5 bara. The temperature profile along the catalyst bed is recorded for each setting. The hot spot temperature is in the range from 400° C. to 650° C., depending on the experiment. Hydrogen is supplied in excess from the beginning in a molar ratio of H₂:O₂ in the range from 2.1 to 2.8.

The total butane-butene conversion(C4 conversion) is determined as follows:

$${X}_{C 4} = \frac{{\dot{η}}_{{l}_{butane}} + {\dot{η}}_{{l}_{butadiene}}}{{\dot{η}}_{{i}_{butane}}^{0} + {\dot{η}}_{{l}_{butadiene}}^{0}}$$

where \dot{n}ᵢ is the molar flow at the outlet and \dot{n}ᵢ⁰ is the molar flow at the inlet of the reactor of component i.

The yields to form butene, CO and CO₂ are based on the starting materials butane and butene and are calculated as follows.

$${Y}_{i} = \frac{1}{{μ}_{i}} \frac{{\dot{η}}_{i} - {\dot{η}}_{i}^{0}}{{\dot{η}}_{{i}_{butane}}^{0} + {\dot{η}}_{{l}_{butadiene}}^{0}}$$

where i refers to CO, CO₂ or butene and μᵢ is the stoichiometric coefficient, μᵢ=1 for butene and μᵢ=1 for CO and CO₂.

The selectivities are then calculated as follows:

$${S}_{i} = \frac{{Y}_{i}}{{X}_{C 4}}$$

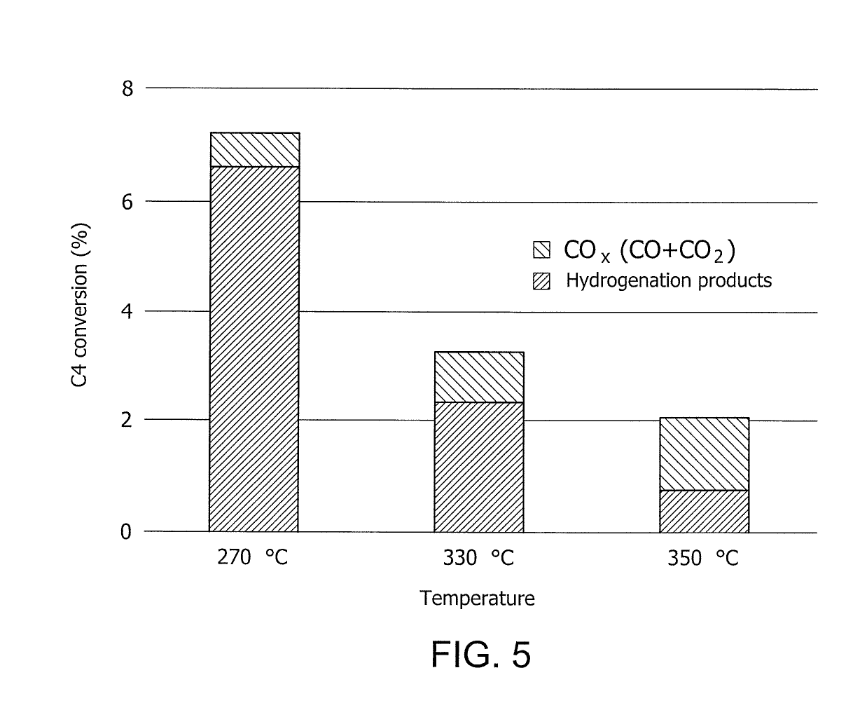

The results were as follows (FIG. 5):

The C₄ conversion at 270° C. was 7.2 mol %, of which 6.6 mol % was hydrogenation products and 0.6 mol % was COₓ (CO+CO₂).

At 330° C., the C₄ conversion was 3.2 mol %, of which 2.3 mol % was hydrogenation products and 0.9 mol % was COₓ.

At 350° C., the C₄ conversion was 2.0 mol %, of which 0.7 mol % was hydrogenation products and 0.3 mol % was COₓ.

If the inlet temperature in the catalyst bed is increased from 270° C. to 350° C., the C4 conversion decreases from about 7% to 2%. At low temperature (<300° C.), the selectivity to butene is >75%, which indicates hydrogenation of butadiene. This hydrogenation decreases significantly in favor of the combustion products with increasing temperature.

### Example 4

Production of the Catalyst:

The catalyst comprises 99.8% by weight of Al₂O₃ (from Axes), SPH-512, bead shape, spheres having a diameter in the range from 2.5 to 5 mm, and 0.1% by weight of platinum and 0.1% by weight of Sn.

100 g of support, 0.25 g of hexachloroplatinic(IV) acid hydrate and 0.19 g of SnCl₂.2H₂O are used for producing the catalyst. The hexachloroplatinic acid is dissolved in 10 ml of water. The SnCl₂.2H₂O is dissolved in a mixture of 17.5 ml of 65.0% by weight HNO₃ and 17.5 ml of H₂O. The two solutions are mixed with stirring and made up to 100 ml with H₂O.

The support is then impregnated with the solution, dried at 120° C. for 30 minutes and subsequently calcined at 500° C. for 3 hours.

### Example 4.1

### Using the Abovementioned Catalyst:

In the reactor described in example 1, a gas stream consisting of 3% by volume of O₂, 8.0% by volume of butadiene, 2.0% of butane, 20.0% of water vapor with balance nitrogen is introduced into the reactor. The catalyst bed consists of 75 ml of undiluted catalyst. The total volume flow is 800 standard l/h. The admission pressure was kept constant at 1.5 bara. Hydrogen is introduced into the gas stream in the line upstream of the reactor (at an H₂:O₂ volume ratio of 2.5:1).

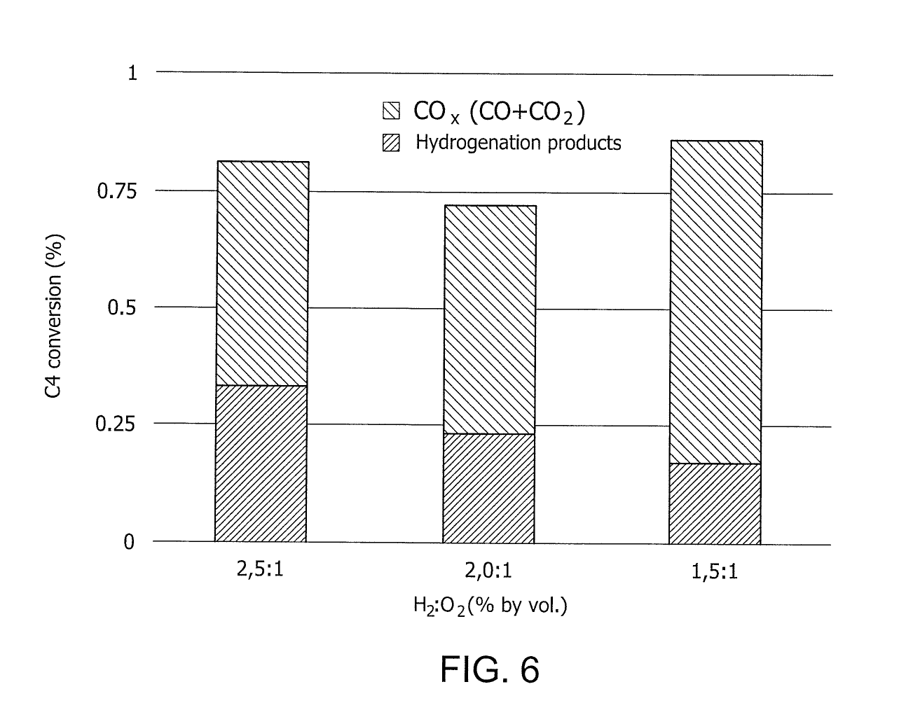

Under the test conditions, no oxygen could be detected during the entire operating time of 200 hours. The inlet temperature of the catalyst bed is 390° C. and the hot spot temperature is 580° C. The butadiene loss is 1.16 mol %. 0.56 mol % thereof is COₓ (CO and CO₂) and the balance (0.60 mol %) is hydrogenation product (butenes) (see FIG. 6).

### Example 4.2

### Using the Abovementioned Catalyst:

In the reactor described in example 1, a gas stream consisting of 3% by volume of O₂, 8.0% by volume of butadiene, 2.0% of butane, 20.0% of water vapor with balance nitrogen is introduced into the reactor. The catalyst bed consists of 75 ml of undiluted catalyst. The total volume flow is 800 standard l/h. The admission pressure was kept constant at 1.5 bara. Hydrogen is introduced into the gas stream in the line upstream of the reactor (at an H₂:O₂ volume ratio of 2.0:1).

Under the test conditions, no oxygen could be detected during the entire operating time of 200 hours. The inlet temperature of the catalyst bed is 395° C. and the hot spot temperature is 600° C. The butadiene loss is 1.32 mol %. 0.65 mol % thereof is COₓ (CO and CO₂) and the balance (0.67 mol %) is hydrogenation product (butenes) (see FIG. 6).

### Example 4.3

### Using the Abovementioned Catalyst:

In the reactor described in example 1, a gas stream consisting of 3% by volume of O₂, 8.0% by volume of butadiene, 2.0% of butane, 20.0% of water vapor with balance nitrogen is introduced into the reactor. The catalyst bed consists of 75 ml of undiluted catalyst. The total volume flow is 800 standard l/h. The admission pressure was kept constant at 1.5 bara. Hydrogen is introduced into the gas stream in the line upstream of the reactor (at an H₂:O₂ volume ratio of 1.5:1).

Under the test conditions, no oxygen could be detected during the entire operating time of 200 hours. The inlet temperature of the catalyst bed is 401° C. and the hot spot temperature is 596° C. The butadiene loss is 1.76 mol %. 1.16 mol % thereof is COₓ (CO and CO₂) and the balance (0.60 mol %) is hydrogenation product (butenes) (see FIG. 6).

1. A process for removing oxygen from a C₄-hydrocarbon stream comprising free oxygen by catalytic combustion, in which the hydrocarbon stream comprising free oxygen is reacted by catalytic combustion over a catalyst bed in the presence or absence of free hydrogen to give an oxygen-depleted hydrocarbon stream, wherein the catalytic combustion is carried out continuously, the entry temperature in the catalyst bed is at least 300° C. and the maximum temperature in the catalyst bed is not more than 700° C.

2. The process according to claim 1, wherein the hydrocarbon stream used comprises from 0.5 to 8% by volume of free oxygen.

3. The process according to claim 1, wherein the hydrocarbon stream comprising free oxygen comprises an amount of free hydrogen which is sufficient for reaction with the free oxygen and/or has this added to it, and the free oxygen is reacted with the free hydrogen.

4. The process according to claim 1, wherein the hydrocarbon stream comprising free oxygen does not comprise any free hydrogen and no free hydrogen is added to it.

5. The process according to claim 4, wherein the free oxygen is reacted with hydrocarbon comprised in the hydrocarbon stream comprising free oxygen or with added methanol, natural gas and/or synthesis gas as reducing agent.

6. The process according to claim 1, wherein at least 80% by volume of the hydrocarbons in the C₄-hydrocarbon stream are C₄-hydrocarbons.

7. The process according to claim 1, wherein the entry temperature in the catalyst bed is from 300 to 450° C.

8. The process according to claim 1, wherein the maximum temperature in the catalyst bed is not more than 700° C.

9. The process according to claim 1, wherein the catalytic combustion is carried out at a pressure in the range from 0.5 to 20 bar absolute.

10. The process according to claim 1, wherein a catalyst comprising at least one noble metal and/or at least one transition metal on a support is used.

11. The process according to claim 10, wherein the noble metal in the catalyst is platinum or a platinum-comprising alloy.

12. The process according to claim 10, wherein the transition metal in the catalyst is Mn, Fe, Ni, Co, Cu, Zn, Sn or a mixture thereof.

13. The process according to claim 10, wherein the support is a-aluminum oxide or zeolite A.

14. The process according to claim 10, wherein the catalyst is present as shaped body having an average diameter in the range from 1 to 10 mm or as monolith, where the monolith can have the catalyst as washcoat on a support structure.
